(19) United States
(12) Patent Application Publication
(10) Pub. No.: US 2005/0038680 A1
(43) Pub. Date: Feb. 17, 2005

(54) System and method for glucose monitoring

(76) Inventor: Kevin Lee McMahon, Dallas, TX (US)

Correspondence Address: Kevin L. McMahon, 1444 El Campo, Suite100, Dallas, TX, 75218-3520, US

(21) Appl. No.: 10/770,946

(22) Filed: Feb. 3, 2004

Related U.S. Application Data
(63) Continuation-in-part of application No. 10/741,967, filed on Dec. 19, 2003.
(60) Provisional application No. 60/435,017, filed on Dec. 19, 2002.

(51) Int. Cl. G06F 17/60; A61B 5/00

(52) U.S. Cl. 705/3; 600/300

(57) ABSTRACT

An system for remotely monitoring a medical condition of a patient includes a unit transportable by the patient and a base system. The transportable unit includes an input device for inputting test information for evaluating a selected medical condition of the patient and a communications device for selectively transmitting test information received from the input device. The base system receives the test information from the communications device of the transportable unit and distributes such test data to at least one member of a medical condition management team.

### CROSS-REFERENCE TO RELATED APPLICATION

This application is a Continuation-in-Part of U.S. Utility application Ser. No. 10/741,967 filed on Dec. 19, 2003 entitled “System and Method for Glucose Monitoring” by inventor Kevin Lee McMahon, currently pending, which claimed the benefit under 35 USC 119(e) of U.S. Provisional Application Ser. No. 60/435,017 filed on Dec. 19, 2002.

### FIELD OF INVENTION

The present invention generally relates to a system and method for obtaining data from external as well as implanted biometric and drug delivery devices including glucometers, insulin pumps, pedometers, accelerometers and other data-enabled instruments relevant to the care of diabetes, and transmitting such data from remote locations providing a highly accurate progression of the patient's glucose, exercise, insulin, carbohydrate intake and other levels for more effective medical treatment.

### BACKGROUND OF THE INVENTION

Affecting as many as 16 million Americans, diabetes is characterized by abnormal levels of sugar in the bloodstream, resulting from defects in insulin production and/or insulin action. A degenerative condition, diabetes causes sugar to build up in your blood and can lead to serious health complications such as heart disease, blindness, stroke, kidney failure and limb amputation.

A healthy diet is just as important as taking insulin or glucose tablets. A low fat, low sugar diet containing plenty of starchy foods and fruit and vegetables helps to stabilize blood fat and blood glucose levels and control weight.

Low-income individuals are the most at-risk group suffering from Type 2 diabetes. One American study, for example, discovered that among low-income earners, 16.1 percent of men and 21.1 percent of women had diabetes, compared to 6.2 percent and 4.0 percent respectively among upper income earners. American Indians had the highest incidences in the world —47.6 percent of men and 48.9 percent of women.

Blood sugar testing is an integral part of diabetes management. Testing helps patients monitor diabetes and make adjustments in their diet and exercise regimen as needed. The goal is to keep blood sugar levels as close to normal as possible. In doing so, the patient can delay or even prevent many long-term health problems caused by consistently high (hyperglycemia), low (hypoglycemia) and the wide swings in blood sugar levels.

In the past, a diabetic patient who needs to monitor and control blood glucose levels typically carried the following paraphernalia: (1) a supply of disposable lancets, (2) a reusable lancing device which accepts the lancets, (3) an electronic glucose meter (glucometer), (4) a supply of disposable glucose test strips for the meter, and (5) tools for insulin injection (insulin, disposable hypodermic needles, and a syringe). The patient typically carries these items in the form of a kit, which may also contain (6) a variety of control and calibration strips to assure the accuracy of the meter and the measurement.

After blood has been transferred to the test strip, the glucose meter then measures the blood glucose concentration (typically by chemical reaction of glucose with reagents on the test strip). Such blood glucose measurements permit the diabetic to manage his glucose levels, whether that is to inject a corresponding dose of insulin (generally Type I diabetic) or using a protocol established with his physician to modify his diet and exercise (Type I or Type II diabetic). Used lancets and test strips are removed and discarded (or kept for subsequent disposal in a hazardous waste container kept elsewhere). Any extra blood is cleaned from the equipment and the wound site, and all pieces of apparatus are stored for future use. The entire process usually takes a few minutes.

From this point, patients have some form of agreement with their diabetes team as to logging and periodic communication of the glucose readings, insulin dosing, and other comments pertinent to the diabetes management regimen. These handwritten “logs” are then faxed to the endocrinology staff or brought with them to their semi-annual or quarterly status checkups with their endocrinologist.

Most patients, however, fail to adequately log and communicate this data, if they keep a log at all, until a critical moment is at hand. Examples of these situations are calling in to get direction regarding “out of control” blood sugar levels or in the doctor's office during the quarterly check up. This need for information is a bottleneck to effective diagnosis and prescription. Even when used, these personal logs are lacking in their precision, timeliness, and sometimes readability, which can make the task of diagnosis and prescribing of changes to the standing protocol difficult.

There have been several attempts to close these gaps in communication and self-management using technologies which include handheld computers, desktop personal computers (“PCs”), internet connectivity, web-based applications, and specialized glucometers that physically integrate with Personal Data Assistants (“PDAs”).

For example, U.S. Pat. No. 5,899,855, issued on May 4, 1999 to Stephen Brown discloses a modular self-care health monitoring system employing a compact microprocessor-based unit such as a video game system of the type that includes switches for controlling the device operation and a program cartridge. The program cartridge adapts the microprocessor-based unit for operation with a glucose monitor. The microprocessor-based unit processes data supplied by the glucose monitor to supply data on the microprocessor-based unit or separate display monitor. The system then transfers the data to a remote clearinghouse that in turn transfers the data to a healthcare professional via facsimile transmission.

Likewise, U.S. Pat. No. 6,144,922 issued on Nov. 7, 2000 issued to Douglas et al. discloses an analyze concentration information collection system and communication system. This invention is described as a two part device including a monitoring instrument and a communications module that rely on each other to generate test data and to forward to an external personal computer or via modem across the internet to an electronic bulletin board.

U.S. Pat. No. 6,427,088 issued on Jul. 30, 2002 issued to Bowman, IV et al discloses an implanted medical device (e.g. infusion pump) and an external device communicate with one another via telemetry messages that are receivable only during windows or listening periods. Each listening period is open for a prescribed period of time and is spaced from successive listening periods by an interval. The prescribed period of time is typically kept small to minimize power consumption. To increase likelihood of successful communication, the window may be forced to an open state, by use of an attention signal, in anticipation of an incoming message. To further minimize power consumption, it is desirable to minimize use of extended attention signals, which is accomplished by the transmitter maintaining an estimate of listening period start times and attempting to send messages only during listening periods. In the communication device, the estimate is updated as a result of information obtained with the reception of each message from the medical device.

### SUMMARY OF INVENTION

The inherent simplicity and low cost of the present invention is what makes it so attractive to clinicians and diabetics. Non-technical users can utilize the present invention with absolute minimal training. In addition, even in the case of the patient who only uses the health management device component of the system provides an invaluable window to the medical profession that will enable proactive patient disease management thereby contributing greatly to the reduction in healthcare costs due to unforeseen complications that are widely known and attributed to diabetes.

Additionally, the remote aspect of this invention is a critical enhancement to such things as the closed loop artificial pancreas as a link between the prior arts that emphasize only the short-range telemetry. A primary use of the invention would be long-range, remote telemetry for a remote monitoring, command and control system.

Moreover, because lifestyle has a direct relationship with the localized time of day, transitions between time zones must be managed and accounted for based on individualized algorithms to determine the transition plan between testing, dosing, carbohydrate intake, exercise, etc. The prior art technologies fail to automate time management and synchronize standards of time as it relates to delivery system scheduling and data marking. The present invention relies on external standards of time to account for the impact of patient mobility, in this case, as it relates to societal imposed standards of time (e.g. Greenwich International Time Zones). This aspect of data cleansing is especially important with a disease such as diabetes due to the direct relationship with meals and carbohydrate intake as well as sleep and exercise.

Further, the present invention enables the development, testing and invocation of various predictive algorithms used for identifying optimizations within the prescribed protocol or new prescriptions. The system may be used to automate the analysis, notification, recommendation, authorization and implementation of the recommended changes in a secure, controlled automated feedback loop system for chronic disease management. Due to the critical nature of the established protocol and the dependence on technology and imperfect techniques and systems, a remote monitoring, command and control approach is essential to the safeguarding of the individuals utilizing aspects of standard disease management systems. This becomes especially important as advancements in technology bring about the experimentation and deployment of expert systems but with only localized monitoring, command and control systems. The inventions described herein attempt to address this critical limitations of the prior art.

Integrating low cost wireless devices using passive data collection methods into the practice of healthcare will add value by helping to overcome the dependencies on human intervention to record and share information in a timely fashion. This will ultimately help to decrease costs, increase efficiency and provide peace of mind during times of separation between those with actual or perceived responsibility for other's care and the chronically diseased patient. These mobile computing devices will transform data into timely, valuable information previously only available at the point-of-care.

The foregoing outlined some of the more pertinent features of the present invention. One should construe these features as merely illustrative of some of the more prominent features and applications of the invention. One may obtain many other beneficial results when applying the disclosed invention in a different manner or modifying the invention as described. Accordingly, one may recognize other features and a fuller understanding of the invention when referring to the following Detailed Description of the Preferred Embodiment

### BRIEF DESCRIPTION OF DRAWINGS

For a more complete understanding of the present invention, and the advantages thereof, reference is now made to the following descriptions taken in conjunction with the accompanying drawings, in which

FIG. 1A depicts an example of a medical apparatus of the present invention utilizing a single microprocessor to perform both the intelligent device polling logic as well as the communications function;

FIG. 1B is a high level functional block diagram of a representative network-based system embodying the principles of the present invention;

FIG. 2 depicts an example of a medical apparatus of the present invention utilizing a second microprocessor to perform the intelligent device polling logic whereas the third party communications processor has only that primary function and thereby relies on the second microprocessor to perform complex processing routines;

FIG. 3 depicts an example of a medical apparatus of the present invention utilizing a second microprocessor to perform the intelligent device polling logic whereas the third party communications processor has only that primary function and thereby relies on the second microprocessor to perform complex processing routines. In addition, this configuration addresses advanced data management techniques and enables premium interactive services via the introduction of a user interface and data input mechanism;

FIG. 4 depicts an example of a medical apparatus of the present invention utilizing a second microprocessor to perform the intelligent device polling logic whereas the third party communications processor has only that primary function and thereby relies on the second microprocessor to perform complex processing routines. In addition, this configuration addresses advanced data management techniques and enables premium interactive services via the introduction of a standalone third party handheld computing device. In this configuration, the PDA is able to synchronize with the case and take advantage of its communications capabilities. Also, by using the connection point usually reserved for data synchronization as the communications port via the communications capabilities of the case, limited expansion slots in the PDA can now be simultaneously used for other peripheral device componentry such as additional memory cards, digital photography, etc . . . . ;

FIG. 5 depicts a high level block diagram of one particular network based medical condition management system embodying the principles of the present invention;

FIG. 6 is a conceptual diagram illustrating typical operations supported by the server, IVR server and computer of FIG. 5;

FIG. 7 is a high level block diagram emphasizing one particular set of communication links between each individual patient management system and the server of the system of FIG. 5;

FIG. 8 is a sequence diagram describing a typical exchange of information between a given patient management system, in this case a glucose meter, and server of the system of FIG. 5;

FIG. 9 is a representative sequence diagram illustrating the configuration of a given patient mobile unit by the server;

FIG. 10a illustrates a exemplary Triage Plot in which Standard Deviation and Average Blood Sugar are plotted over the 14 most recent days on a rolling basis;

FIG. 10b illustrates a Community Plot, which is a graphical non-patient identifiable representation of the universal database over 14 most recent days on a rolling basis;

FIG. 10c shows a graphical representation of the log data log for the selected patient;

FIG. 10d illustrates the Mouse-over of Patient Highlight feature, which allows easy identification from the logged data of any data point on the plot;

FIG. 10e is an exemplary Modal Day Plot, which shows readings for a single patient from the last 14 days in a modal day view;

FIG. 11 illustrates a home monitoring and goaling system according to another embodiment of the principles of the present invention; and

FIG. 12 illustrated the embodiment in which the biometric sensors and patient mobile unit are either worn on-body or kept close to the body.

### DETAILED DESCRIPTION OF THE INVENTION

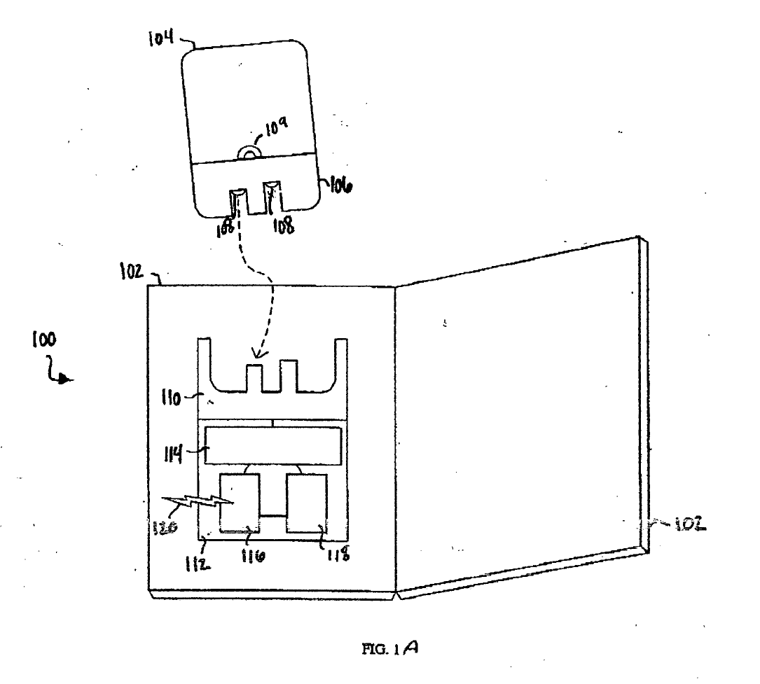

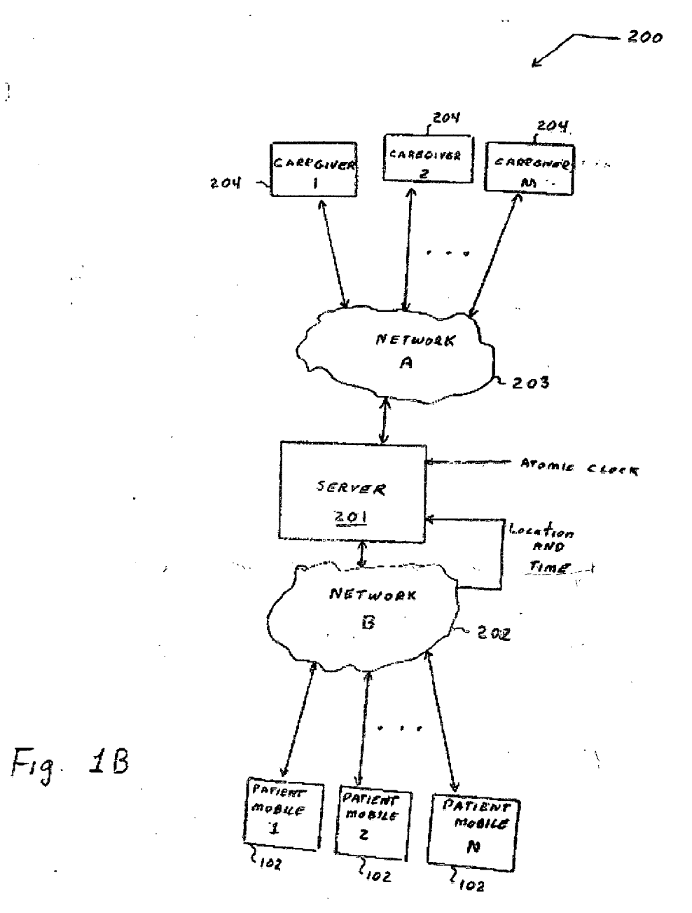

The principles of the present invention and their advantages are best understood by referring to the illustrated embodiment depicted in FIGS. 1-12 of the drawings, in which like numbers designate like parts.

The particular values and configurations discussed in these non-limiting examples, however, can be varied and are cited merely to illustrate an embodiment of the present invention and are not intended to limit the scope of the invention.

FIG. 1 depicts an example of a remote, real-time diabetes management system (mobiles) 100 including a handheld case 102 with a nexus between communications components and biometric devices that can be integrated using a two-plate configuration. A device connection plate 106, has a multitude of configurations necessary to provide for easy and logical placement and storage of an individuals data-enabled disease management tools. Specifically, device connection plate 106 provides a hardwired interface between a selected biometric device 102 and a corresponding plate 110. These devices are situated in such a way so as to simultaneously invoke polling of biometric device 102 via a completed physical connection at the same time that they are replaced into their dedicated home within health management case 102. For example, in the case of diabetes, biometric device 102 is a glucometer, which can come in different sizes and different data-port locations and interface technologies (e.g. stereo-plug connectors, infra-red, optical recognition, wireless, audio recognition, etc . . . . ). Connection plate 104 provides connections 108 which wire to the specific terminals of biometric device 102 and also mate with plate 110, such that a physical and electrical interface between biometric device 102 and management systems 100 is supported. Likewise, the inventory of biometric devices 102 varies greatly by patient thus creating a multitude of patient-specific device storage options that may include, among other things: glucometers; insulin pumps and/or other insulin injection devices; pedometers and/or other exercise/activity measuring devices including accelerometers; and thermometers and/or other temperature sensing devices.

FIG. 1B is a high level functional block diagram of a representative network-based system 200 embodying the principles of the present invention. System 200 is centered around a server 201 operated by either a public or a private entity. In addition to providing overall system control, server 201 receives and collects biometric information from a corresponding set of N number of patient mobile (management) units 100, three of which are shown in FIG. 1B for reference. The biometric information generated by patient mobiles 100 is transmitted by server 201 through a network 202, which is preferably a wireless network, although network 202 could also be a combination of wireless and hardwired network components. In the illustrated embodiment, patient mobiles 102 transmit via a wireless link to network 202 for further transmission to server 201. In a fully wireless environment, network 202 is a wireless wide area network supported by a commercial provider, such as Skytel, Weblink Wireless, or the like. Alternatively, network 202 may include access points, such as IEEE 802.11x access points which receive wireless data from patient mobiles 102 in the area of given access points and subsequently transfer the associated biometric data to server 201 via a hardwired connection.

Biometric data collected by server 201 from patient mobile units 102 is distributed to one or more of M number of care givers 204 through network 203. Network 203 is preferably a hardwired interconnection through a private network, such as a private wide area network, or a public-based network, such as the Internet or the World Wide Web. Individual care givers can then utilize their own individual automated risk-based population stratification schemes for identifying particular patients, which require particular attention. Caregivers generally include doctors, nurses, school nurses, hospitals, clinics, family members and relatives forming a team supporting the care of a corresponding patient.

Server 201 receives time and location information from each patient mobile 102, allowing the corresponding care giver 204 the ability to monitor the timeliness of the patient's testing and monitoring activities. In the illustrated embodiment, server 201 controls the system timing, in conjunction with networks 202 and 203, from a national atomic clock or similar standardized time base.

Advantageously, utilization of system 200 does not require a modification of patient behavior. In other words, since system 200 is transparent to the individual patient, the patient need not perform any additional task, (e.g., connecting to the network, contacting the caregiver directly, etc . . . . ) other than those already prescribed by the doctor for use of the given biometric device 104. Further, system 200 supports event-based and trend-based triggers which allow healthcare providers to intervene in response to test results which cross a given threshold or tend towards a threshold. For example, a test of blood sugar below a given level may trigger a prompt (either automated, rules based, or human) to the patient (via telephone, email, etc . . . . ) to perform a retest or take other appropriate action.

Management system 100 also includes an electronics board 112 having a conventional radio-frequency (RF) transceiver 116, a microprocessor 114, responsible for managing the commands and logic of the RF transceiver 116, and a power supply 118.

Wireless connection (transmission) may occur by any number of means. However, in the preferred embodiment of the invention, a radio connection adds to the simplicity of use by removing the need to physically connect to another device in order to share information resident in the management system 100. This connection can be short range, as in the case of an IEEE 802.11x wireless connection to a wireless access point, or long range, as in the case of cellular and paging networks. In any case, the point of transmitting is to enable the sharing and distribution of data and information. Additionally, this transmission and reception capability allows for remote diagnostics of the device componentry and the electronics themselves. The role of transmitting this data is shared by a multitude of computers. The goal of transmitting this data is to facilitate timely and appropriate communication within an infinite number of public and proprietary processes.

Power supply 118 can be of any source including replaceable and rechargeable battery, solar cells, etc . . . . it is simply the source of power to drive the electronics within the case and not necessarily used to drive the third party devices although that is one option.

Interface plate 109, coupled to biometric device 104, by plate 106, senses physical connections with plate 110. Integration device 109 is a part of plate 106 and universally applicable to any third party biometric device 104. This is primarily one of many mechanisms management system 100 employs that abstracts behavioral dependency from the device polling and transmission process. By placing a device or connection into plate 106, the sensor is physically affected in one or many ways to acknowledge a change in state which then invokes various device polling routines which among other things, checks for new data in third party biometric device 104. These integration sensors 109 can also be used to verify connection between the componentry of the case as a means of troubleshooting the system.

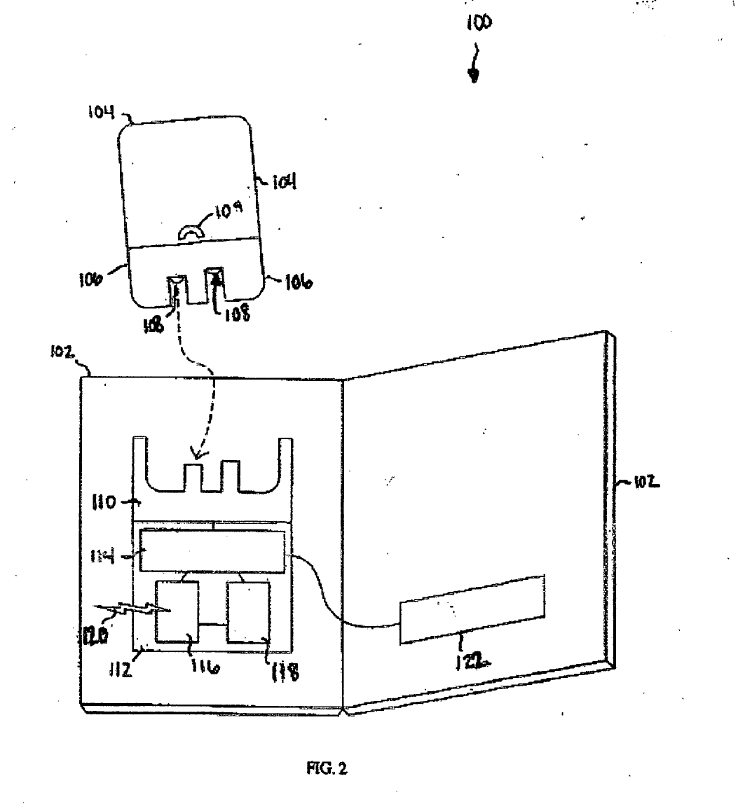

In the alternate embodiment of management system 100 shown in FIG. 2, a second microprocessor 122 is used in addition to the RF board processor 114 when additional processing power is required. One example of when this second microprocessor 122 would be utilized to manage complex polling routines that would check for data and to intelligently manage the transmission decision. This is a different function than what the RF board processor 114 is tasked to do, as it operates with minimal intelligence and simply reacts to inbound and simple outbound transmissions. To support a reasonable battery life for the unit, the user of the case 102 for the purpose of sending real-time data would prefer the second microprocessor option. This allows the additional processing power to intelligently manage the polling and transmission with the role of also optimizing the operation thus extending the battery life.

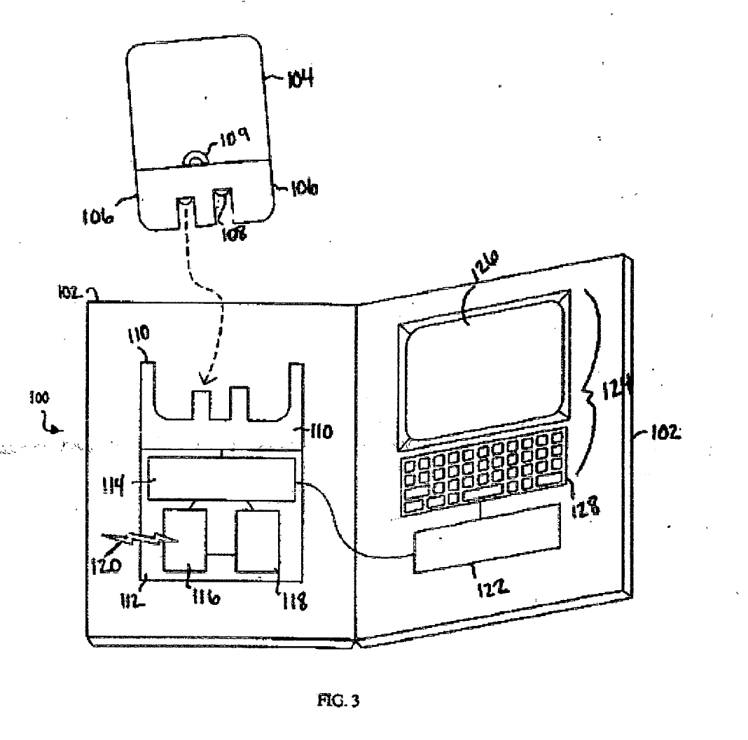

The alternate embodiment of FIG. 3 includes an optional user interface 124, which can be comprised of both an input technology 128 as well as an output technology 126, either combined as a single unit or separately as shown here.

In the preferred embodiment, the user interface output mechanism 128 would typically be a sensory unit that would be meaningful to one's senses including sight, hearing, etc . . . . This is typically an LCD type screen with text, symbols, colors or the like as well as audio of some kind.

In the preferred embodiment, the user interface input mechanism 126 would typically be a sensory unit that would be meaningful to one's actions and abilities including speech, typing, button depression, etc . . . . This is typically a keyboard, drawing screen, audio converter or recorder, specialized buttons with aggregated meanings (e.g.—consumption of small, medium or large meal which would have further definition elsewhere in the system).

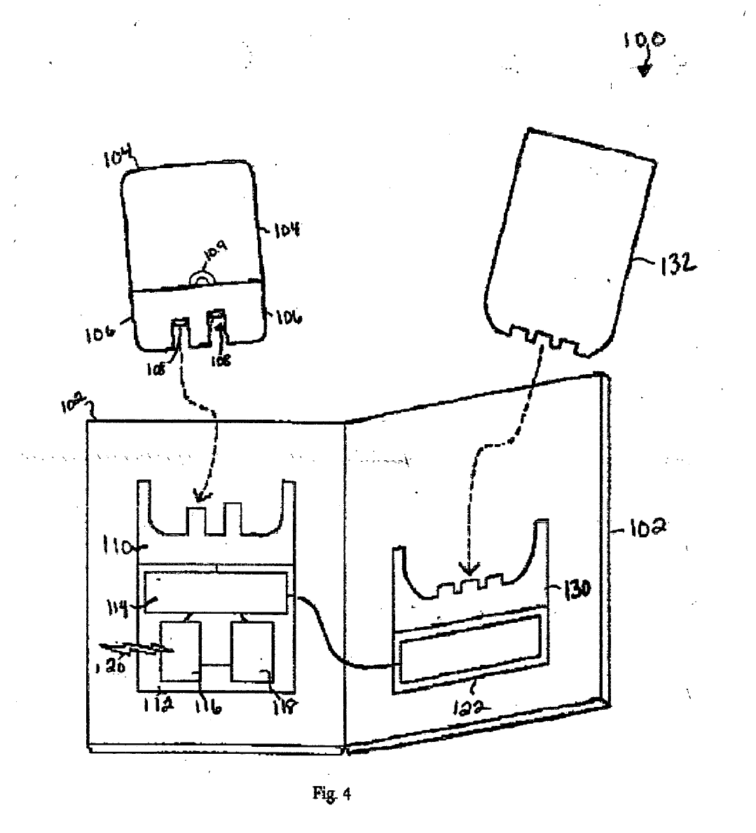

The embodiment of management system 100 shown in FIG. 4 includes a third set of interconnection plates 130 and 131, similar in function to plate 106 and to plate 110. This feature allows for the flexible yet planned integration of third party electronics 132 such as a personal digital assistants or micro/handheld computing devices. Such a device would contain its own user interface(s), microprocessor(s), power supply. However, by integrating through this planned docking station allows for the opportunity of shared services such as power recharging, processing power and the exchange of information, synchronization, programming, etc . . . . Third party electronic device 132 is a self-contained computing device such as a PDA, digital music player, etc . . . . with significant data management application capabilities that one would use independent of the case and for purposes other than biometric diagnostics.

The communications connection plate 106, has a multitude of configurations necessary to provide for easy and logical placement and storage of an individuals preferred communications requirements. Electronics board 112 focuses on allowing a multitude of various third party communications modules including network specific communications boards. The preferred network type is of, or having to do, with radio or cellular transmission including any format or protocol. Examples of these wireless protocols are Reflex, Mobitex, GPRS, GSM, CDMA, and 802.11x of any format. Additional communications ports might include non-wireless means and specific physical requirements for communications via USB, Ethernet, IEEE 1394.x where x may equal any combination of letters or numbers, or any other present or future communications protocol and its physical connection requirements.

Management system 100 provides for several integration methods and physical ports designed for transparent technical and behavioral access to the biometric device data. In order to facilitate the notion of transparency and abstracting human dependencies from the act of data harvesting from the biometric devices, the following techniques and physical components are described that all relate back to the intelligent software housed on either of the aforementioned microprocessors. Since not all data-enabled biometric devices have the same requirements for data uploading by/to an external microprocessor, the intelligent software within management system 100 must have device specific preferences and rules for ensuring the most timely and accurate polling and appropriate biometric device-specific techniques without requiring a constant connection. In the preferred method the software will allow for the electrical sensing of changes in the electrical properties of the connection. Further, the software should allow for timing or chronological scheduling based on initial parameters set by the user and later driven by either human-designed intervals or, as a preferred method, automated timing intervals established by the software's historical view toward the presence of new device data. This is yet another actualization of the intelligent software abstracting human intervention and dependency.

Device and location specific, spring-loaded plates 104, 106 are yet another mechanism that can provide a passive, intelligent mechanism to understand that a device has been both removed from the case as well as replaced into its dedicated location within the case. Again, the intelligent software can be designed with device specific routines and rules that take this in/out awareness into account when determining the appropriate time to poll the respective device for new data. Human intervention in the form of depressing a button or any other simple technique for invoking the device polling function. Transmission and other data management functions would be automatic past that initial point of human intervention.

In the preferred embodiment of the invention, there is intentionally no user interface on management system 100 for enabling human intervention. An example of “user interface” would be an LCD screen or computer-generated speech for facilitating one-way communications as well as the preceding plus a communications input mechanism such as a text keyboard or audio recorder for facilitating two-way communications. This is done in order to: eliminate human error; reduce support costs that come with more complex, interactive wireless devices; lower the cost of manufacturing the device; and reduce the likelihood of theft by severely limiting the role and perceived value only to those familiar with the exact purpose and function of the device. An exception to this would be simple indicators for indicating successful transmission or function completion such as audio tones, temporary visual lighting nodules (e.g. LED indicators of green, red, yellow, etc . . . . ).

In the embodiment of the invention shown in FIG. 3, user interface 124 can be a priority function of the device. However, it is very important to distinguish the importance the health management case 100 both with and without the characteristics that come with the user interface functionality. User interface 124 is a premium feature geared only toward those with a mind toward aggressive disease management. This notion of a user interface can range from case-specific LCD screens and an embedded text input keyboard, to a docking station for a text input device either with or without external communications capabilities, to a fully functioning personal digital assistant which would require an accompanying docking station for the computing device in the context of the aforementioned device connection plate 104, the device connection plate. The implementation of this docking data port may be as described within device connection plate 104 or as a separate, plate 130 (FIG. 4) designed as a docking station for third party computing and communications device as in the case of the PDA or Cell Phone or other textual and communications device.

Remote communications of the biometric device data 104 is passively and intelligently transmitted to a remote computer, in system 200, server 201. In the preferred embodiment, this communication uses a third party's private wireless network however any means of transport is relevant to the data transmission.

Software intelligence to govern the data access and data management may reside both onboard either of the case-local microprocessors or on board any number of remote computers. A combination of user defined and computer-derived rules govern the flow of data and translation into information. This subsequent information may be processed and reside either together or apart both locally and remotely or in any combination thereof. Preferably, server 201 supports an automatic risk-based population stratification scheme which allows a caregiver an “at-a-glance” evaluation of a patent practice encompassing a large number of patients. The system (server) software algorithms will determine optimization in terms of the location-specific processing limitations, usage requirements and transmission costs as it relates to the appropriate sharing of data and information keeping in mind the managed cost limitations of the system. The system also includes specialized tools for providing easy analysis for any number of patient's disease state and to facilitate the analysis, determination and recommendation of lifestyle changes to a prescribed or actual disease management protocol.

Therefore, due to the nature of the invention, time is managed separately within the many disparate subsystems within the overall system 200. First, time may be managed within any invasive bio-implant, then within any short range external bio-implant communication system, again, within an external biometric device, then within the proposed invention acting as the remote telemetry communications module, again within a handheld computer used by the subject, again within a circuit-switched communications device, again within the initial wireless base station network element of the wide area wireless network, again within the various gateway computers managed by the operator of the wide area wireless network, again within the gateway computers managed by the remote biometric device and invasive bioimplant monitoring system computers, as well as a myriad of additional keepers of time. What is critical is the availability of relevant data from the myriad keeper's of time and the logic to discern the “best” indication of time. This is especially critical when one chronic disease patient crosses time zones and therefore due to lifestyle modifications imposed by one who participates in society, behavior changes accordingly. This is obvious when one considers meal times and the associated intake of carbohydrates that will affect the physiology of the chronic disease patient as well as the prescription regimen for pharmaceutical or natural drugs used in managing the chronic disease.

One such tool is known as the “Triage Plot.” This graphical depiction allows any user to easily identify a group subset as being in any number of tiered chronic conditions relative to a standard or to the peer group being included in the analysis. The physician's practice must have this capability to quickly identify, at-a-glance, those patients in a chronic state or trending toward a chronic state using a multitude of discriminating parameters. Likewise, it is essential that the user of the tool be able to dynamically modify their parameters used to identify the chronic pool, easily, within a single session of the remote analysis. An example of these parameters may be the establishment of a patients historical blood glucose average over some defined period of time. This average should be normalized prior to plotting as the user pool come from a large group of patients all of whom have their own unique definitions of “Normal,” “High”, and “Low”.” Normalization can be obtained by plotting the average as a percent within the patient-specific range for the appropriate categorization of low, normal, high. This normalization can be performed for all subjects identified within the patient-comparison or patient-relevant groupings. These groupings may be defined by the user as all patients within a given practice, all similarly aged patients within a population, basically, an infinite number of parameterizations. This data point can then be plotted on one of the axis. An example use for the other axis may be a measure of resource utilization captured by the user of the Triage Plot. One such parameter can be the number of calls logged by the physician's office or some other measure of a patient's specific resource utilization. These two data points would then determine the location of the Plotted patient and would indicate the relationship between relative chronic disease state and office resource utilization. Once this plotting is completed for the group of selected individuals, the user of the tool has an easily understandable chart of information that indicates the priority patients for proactive disease management. Since the information is obtained in a timely fashion, physicians and their staff now have the opportunity to exercise Proactive patient disease management instead of Reactive patient disease management. There are an infinite number of parameters and uses for this plotting mechanism. What is claimed specifically is the method for promoting the visual segmentation of a population so as to enable the user of the information management tool to make quick decisions based on timely information across a diverse set of data sources and to be able to act on this information in a manner consistent with the objectives of parameter selection. In the example, the objective is to increase resource utilization by prioritizing chronic patients relative to both their high resource utilization as well as a lack or inappropriately low resource utilization.

Yet another aspect of this system is the design toward accessing third party developed and managed algorithms for predictive disease management as well as making the stored data available to such third party predictive disease management algorithms. It is not possible for a limited number of resources or individuals to develop the analysis equations that would produce the most accurate feedback recommendations for something as varied and diverse as the management of diabetes. Therefore, it is only through establishment of a data and information clearinghouse with actual meta-data that the scientific community can have access first to testing various hypothesis and to subsequently place into a reliable automated communications role, the proven and reliable advice for promoting self-management through automated recommendations for lifestyle changes.

As part of the function of creating a clearinghouse of diabetes relevant data, it must be understood that a large population of diabetics and their care teams will always have diverse requirements and preferences when it comes to their preferred tools. As such, it is important to allow for personal tool selection and to also provide a non-intrusive mechanism for harvesting the data and subsequent patient-defined information and to make this data/information available to the aforementioned clearinghouse of meta-data. It is through the clearinghouse that peer group analysis can easily take place whether this is by a physician's office, a medical research team, or simply a collaborative group of patients who wish to share and compare their data and information. This aspect of the system provides for that level of abstraction between personally selected and utilized day-to-day tools and the ability for a community to take advantage of the experience of its respective members. This design is actualized in this area of diabetes management and other disease management groups by allowing for a software agent that can be either co-located with the any number of an individual's third party data management applications or positioned remotely providing reliable remote communications and access to the third party diabetes data management application. This communication can be either a one-way harvesting of the data/information or can be a synchronized two-way function providing that the developer of the third party localized diabetes data management application is able to function with the receipt and subsequent data handling requirements of the non-patient specific or enhanced information from the meta-data clearinghouse.

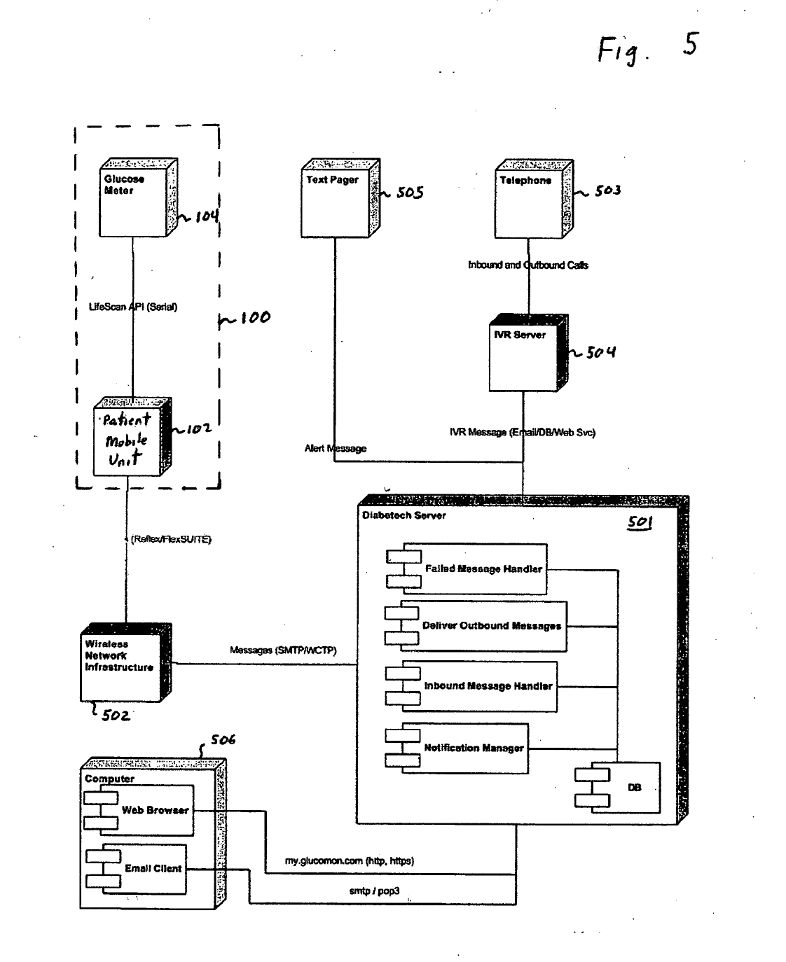

FIG. 5 is a high level block diagram of one particular network based medical condition management system 500 embodying the principles of the present invention generally described in FIG. 2. For purposes of discussion, it will be assumed that system 500 is being utilized for the treatment of diabetes, although the principles of the present invention are applicable to the treatment and management of a wide range of chronic ailments.

System 500 is based upon a server 501, which implements, in hardware and software, a handler for delivering outbound messages, an inbound message handler, an alert manager, and a data base (DB). Specific operations of the server 501 will be discussed further below; however, generally, server 501 supports overall system administration and operates in conjunction with a set of patient mobile units 102 (previously described) in a collaborative fashion to provide the automatic input and analysis of patient data. Each patient mobile unit 102 communicates with a biometric device 104, which in this example is a glucose meter, via the appropriate data link, for example a LIFESCAN API serial interface. In turn, server 501 communicates with each patient mobile unit 102 utilizing a wireless protocol such as Reflex or Flexsuite and smtp/wctp messaging across a wireless network infrastructure 502.

Server 501 also exchanges inbound and outbound telephone traffic from a telephone 503 through an associated interactive voice response (IVR) server 504. Additionally, server 501 can broadcast alert messages, discussed further below, via a wireless link to a conventional text pager 505. A personal computer 506 or similar end-user terminal allows a member of a patient management team to communicate with server 501 via a global computer network, such as the Internet or World Wide Web. In system 500, computer 506 supports a web browser for exchanging data in the http or https formats to server 501 through a dedicated website my.glucomon.com. Additionally, computer 506 supports e-mail client software for communicating with server 501 in smtp, pop3 or other messaging protocols.

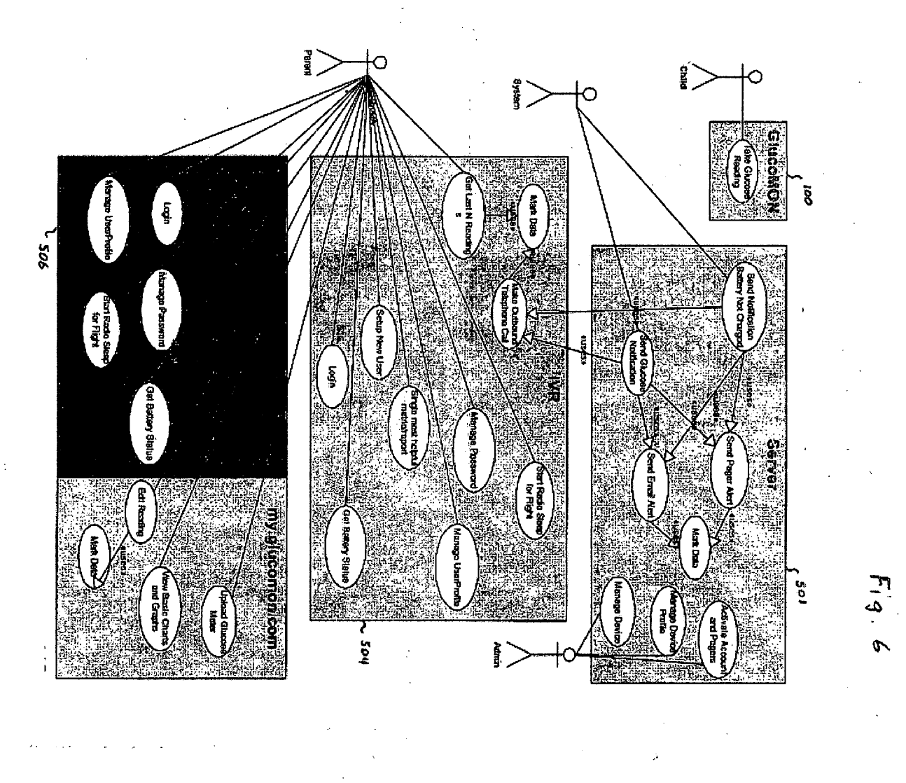

FIG. 6 is a conceptual diagram illustrating typical operations supported by server 501, IVR server 504, and computer 506 of FIG. 5. Among the administrative functions performed by server 501 are device management, device profile management, and activation of accounts and pagers. The device profile management function allows server 501 to configure system 500 to collaborate with a patient and management team through the corresponding patient mobile unit 102. For example, the device profile management function sets up mailing addresses for sending alert messages via IVR 504, text pager 505, and/or computer terminal 506. The device profile management function also sets up the network information controlling communications with mobile unit 102, sets the auto delete glucose meter option, controls the encryption settings, and sets the time zone and auto time settings.

The device management function advantageously allows the data to be not only read from glucose meter 104 of FIG. 5, but also for that data to be erased after that data is successfully downloaded to server 501. This feature is particularly useful with respect to compliance with Federal requirements for patient data confidentiality, since every time test results are received by server 501, those confidential test results are erased from glucose meter 104 to prevent unauthorized download. The device management function also allows data to be erased from patient mobile unit 102 by server 501, as well as allowing server 501 to send control commands to patient mobile unit 100. Server 501 can also determine the battery status for the given patient mobile unit 100 by using the device state management function. (In the preferred embodiment, the patient cannot erase or alter the data stored on patient mobile unit 102, leaving that responsibility solely to the discretion of the management team).

The activate account and pagers function allows new patients and patient management teams to activate corresponding account on server 501, and configure system 500 to communicate alert messages to text pagers and similar appliances. For example, the activate account and pagers function provides for the set up of the proper user identification numbers and passwords.

Server 501 preferrably glucose notifications to members of the management team and/or the patient using an outbound telephone call supported by IVR server 504. Telephone messaging provides the most mobile and flexible technique for establishing the required links between all necessary parties involved under a given set of circumstances. Similarly, when the associated patient mobile unit 100 has discharged its battery, server 501 may send a notification using a similar outbound telephone call via IVR server 504 to the patient and/or management team members. Alternatively, notifications, including notifications of battery status and/or patient glucose level, can be sent by server 501 with a pager alert via text pager 505 of FIG. 5 or through an e-mail alert via computer terminal 506.

A patient mobile unit mark data function allows patient mobile unit 100 to mark particular data which appears to be suspect, such as data which is associated with a suspect or clearly incorrect time stamp. This record is then transmitted and stored at server 501, without affecting the original glucose meter determined time stamp. These suspect readings can be sent via text message or other means to the appropriate member(s) of the team thus providing a simple means of subjective human intervention to either approve, ignore or mark the record with a different time stamp.

IVR server 501 supports interactive voice response communications with members of the patient management team. A log-in function allows for new user set up, including determining a password and a personal identification number (PIN). For example, the password could be generated by concatenating the patient's five digit zip code, four digit year of birth, and six digit PIN number. A managed password function allows for an authorized patient or team member to change the password or recall a forgotten password with the input of appropriate verification information.

A user profile management function supported by IVR server 504 allows authorized management team members to manage the alert messages issued by server 501. For example, the alert management function is used to set the alert destination addresses (e.g., telephone number, fax number, e-mail address) of one or more management team members to which alert messages are to be sent. Constraints can also be imposed on the days of the week and/or start and stop times acceptable for sending a given management team member alert messages. Additionally, the alert message mode can be selected from compliance mode (e.g., all messages sent to a given management team member), exceptions mode (i.e., only selected alert messages sent to a given team member), reminder or prompt mode which contacts various members of the team depending on static or dynamic criteria (e.g.—timed follow-up reminders to prompt actions based on prior data received or missing as in the case of a hypoglycemic test result requiring a retest) or no message mode (i.e., no messages sent to a given management team member).

The start radio sleep for flight option allows a management team member to force the radio receiver within patient mobile unit 102 into a sleep mode. Generally, since radio receivers are not allowed on commercial airline flights, the mobile unit radio receiver must be disabled before flight, as provided by this IVR server 504 function. Additionally, a management team member can monitor the current battery status of the patient mobile unit 100 battery using the get battery status function of IVR 504.

The single most helpful metric/report function allows an authorized management team member utilizing IVR 504 to select the metric data and/or report found to be most useful in analyzing the data from patient mobile unit 102. In particular, this function allows a management team member to customize the report and data to optimize the management of the patient's particular medical case.

As discussed above, server 501 broadcasts battery not charged/low battery status and/or glucose notification messages to one or more management team members through IVR server 504. For example, if a battery low event is received, and no battery full event response is subsequently received within a given time period (e.g., one day), then a battery status notification may be sent to the management team member requesting that the patient mobile unit be charged. Similarly, a notification is sent to one or more management team members if a send battery status command is sent from server 501 to patient mobile unit 102 and no response to the battery command is returned within a given period of time (e.g., one hour).

A glucose notification is made in either the compliance mode and the alert mode. In the compliance mode, all glucose readings for a selected active time window to the management team. In the alert mode, only glucose readings which are out of threshold (high or low) are sent for active time window.

The get last end reading and mark data functions allow a management team member to access any number of sets of results downloaded from patient mobile unit 102. The recalled records can then be marked to indicate specific circumstances under which the test results were taken by the patient. A set of exemplary data markings which can be input via vocal prompts through IVR server 504 are as follows:
To indicate the presence or lack of Ketones, Press 1
To indicate Small, Press 1
To indicate Medium, Press 2
To indicate Large, Press 3
To indicate No Ketones, Press 4

To indicate a Sick Day, Press 2
To indicate Suspected Onset, Press 1
To indicate Medium-type Sick Day, Press 2
To indicate Very sick including loss of fluids, Press 3
To indicate that the patient is feeling better, Press 4

To indicate Exercise, Press 3
To indicate Low, Press 1
To indicate Medium, Press 2
To indicate High, Press 3

To indicate an injection or bolus of Short Acting Insulin, Press 4
Enter a number between 0.00 and 999.00 to record the short acting insulin given, and press the # key

To indicate an injection of Long Acting Insulin, Press 5
Enter a number between 0.00 and 999.00 to record the long acting insulin given, and press the # key
To indicate or update the current total daily basal insulin, Press 6.

Entering a value here will make a change in the basal profile.
Enter a number between 0.00 and 999.00 to record the total daily basal insulin given, and press the # key

To indicate that the insulin pump infusion site has been changed, Press 7

To indicate intake of Carbohydrates, Press 8
Enter a number between 0.00 and 999 to record the amount of carbohydrates associated with this timeframe, and press the # key
Enter 00 to indicate the withholding of normally scheduled carbohydrates, and press the # key

To indicate a suspected influence on the blood sugar level, Press 9
To indicate excitement, Press 1
To indicate tiredness, Press 2
To indicate missed insulin, Press 3
To indicate too much insulin, Press 4
To indicate excessive heat, Press 5
To indicate travel, Press 6
To indicate other medications, Press 7
To indicate unusual changes to your lifestyle or daily schedule, Press 8
To indicate Errors on your meter or problems with your test strips, Press 9

Similar to IVR server 504, a management team member can perform log in password, password management, user profile management, battery status check and a radio sleep for flight functions via computer terminal 506 and the my.glucomon.com website. Additionally, this computer network interface allows authorized management team members to mark data, similar to the IVR marking described above, upload glucose data from the patient mobile unit 102, and view basic charts and graphs. Generally, computer terminal 506 and the myglucomon.com website provide an alternate, albeit less flexible, interface between members of the management team, the patient, and server 501.

Server 501 advantageously supports a number of additional processing options which further increase the flexibility and utility of systems embodying the principles of the present invention. For example, server 501 supports dynamic algorithm management functions in which server 501 analyzes such factors as the results from patient mobile unit 102, changes in patient behavior, changes in treatment regimen, and physical factors, such as patient temperature and carbohydrate intake. From this analysis, server 501 selects from algorithms available from the software development community, for example through automatic download from a network, for use with a particular patient and associated patient mobile unit. 102.

Server 501 also supports virtual—loop feedback mechanisms for collecting information from the corresponding patient mobile units 102. In particular, server 501 operates to collect data and deliver appropriate prompts to the individual patient mobile units 102 for the entry of additional subjective or interactive data. In this fashion, server 501 insures that the patient management team obtains a thorough data collection from the patient, with minimal effort or concern on the patient's part.

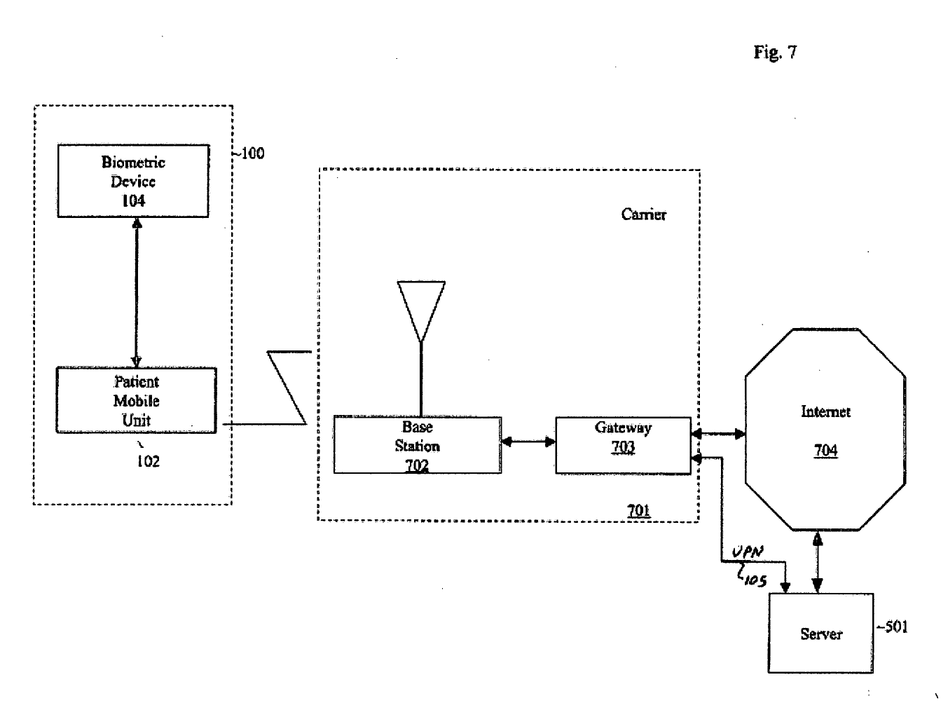

FIG. 7 is a high level block diagram emphasizing one particular set of communication links between each individual patient management system 100 and the server 501 of system 500. In the representative system illustrated in FIG. 7, information exchanges are made between the individual patient management systems 100 and a carrier, such as a Weblink Wireless, SkyTel, or AT&T Wireless, shown generally at 701.

Generally, a carrier 701 communicates with patient management units 100 through a conventional wireless base station 702. Specifically, base station 702 communicates via a conventional network gateway 703 and the internet 704, or similar global computer network, to server 501. In the illustrated system shown in FIG. 7, communications between given patient management system 100 and carrier 701 is established using the Reflex wireless communications protocol known in the art.

Internet connection 704 provides a less expensive, although slower and less reliable means for the exchange of data between gateway 703 of carrier 701 and server 501. For more critical data, an optional virtual private network (VPN) connection between gateway 703 and server 501 is also provided in the system shown in FIG. 7. VPN connection 705 provides higher quality data transmission services, supports better control by server 501 and has increased reliability, although VPN connection 705 will generally be more expensive to implement from a cost and bandwidth point of view.

Messaging between patient management system 100 and carrier 701 preferably utilizes the Wireless Control Transfer Protocol (WCTP) message format, with at least the data payload encrypted in accordance with the AES data encryption standard. Advantageously, the encrypted portion of each WCTP message passes all the way through carrier 701, Internet 704, and/or VPN line 705 to server 501 in an encrypted state. Thus, Federal mandates regarding the maintenance of security and privacy of patient data are not violated during the data transfer.

During transmission of each message, gateway 703 maintained by carrier 701 appends latitude and longitude data in an unencrypted header to the transmitted data packets indicating the location of the given patient management unit 100. From these location data, server 501 can determine the time zone in which that patient management unit 100 currently resides, as such that the data received by server 501 can be appropriately time stamped.

WCTP messages, or other protocol including SMTP, SMS, etc . . . . , received by server 501 from patient management system 100 are decrypted and decompressed to extract the patient data. Server 501 then determines the patient account, and updates and stores the corresponding patient record. Server 501 also applies the rules for generating the alert messages described above.

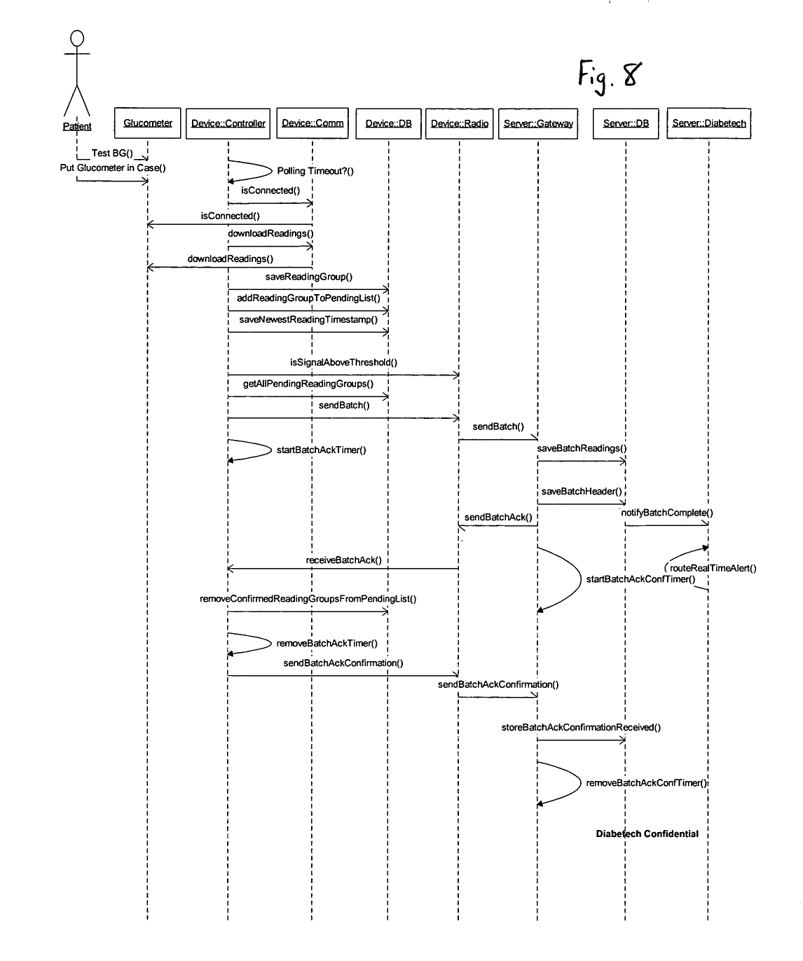

FIG. 8 is a sequence diagram describing a typical exchange of information between a given patient management system 100, in this case a glucose meter, and server 501 of system 500 of FIG. 5.

Microprocessor 114 of the given patient mobile unit 102 periodically senses for the presence of a biometric unit 104 connected to connector 110 of management system 100. In this example, the patient has attached a glucose meter 104 to connector 110 and that glucose meter 104 has been detected. Likewise, the invention may also be embodied within a fixed integration of a glucose sensing technology and the transmission technology. Consequently, a signal is sent to glucose meter 104 and the current set of readings stored within glucose meter 104 are downloaded to mobile unit 102. These readings constitute the results of one or more tests taken by the patient since the last time the glucose meter 104 was connected to mobile unit 102. Specifically, only the delta (difference) between the data stored since the last download from glucose meter 104 is downloaded during the current downloading operation.

Microprocessor 114 then saves the newly downloaded data from glucose meter 104 to a reading group. The reading group includes both the currently downloaded data and all data which are resident in mobile unit 102 but have not as yet been transmitted to server 501. In other words, the new readings taken from glucose meter 104 are stored with any pending data to be sent to server 104. This reading group is then added to the list of pending data to be sent to server 501 when the system is ready. At the same time, the newest time stamp corresponding to the most recently downloaded data is saved.

In the preferred embodiment, all data read from glucose meter 104, along with the appropriate time stamps, is stored in patient mobile unit 102 and stored according to the glucose meter 104 serial number. In one embodiment, after patient mobile unit 102 has stored the reading(s) and after server 501 has stored the readings and confirmed successful storage of the readings, patient mobile unit 102 can send an erase command to the glucose meter 104. For example, this can have the effect of eliminating the presentation of invalid data to the user of the glucose meter 104 as in the case of the glucose meter 104 presenting the simple mean average which may not be statistically valid. Users can however access or schedule the delivery of statistically valid glucose meter 104 generated data from server 501.

Next, microprocessor 114 determines from radio 116 if the communications signal with base station 702 of carrier 701 is above the minimum threshold required for reliable transmission. If the transmission signal is above the required threshold, then the batch of data including the pending reading groups and time stamp data are transmitted from system radio 116 to carrier gateway 702, and in turn on to server 501 where it is stored in the server database. At the same time, microprocessor 114 of patient mobile unit 102 starts a batch acknowledgment timer to define a window in which an acknowledgment of receipt of the batch of data is expected to be returned from server 501.

Upon successful receipt of one or more data points from a patient management system 100, patient mobile unit 102 waits for the return of an acknowledgment signal from the network. As required by the specific patient management rules, server 501 sends a real time alert message via IVR server 504, text pager 505, and/or computer terminal 506 to appropriate members of the patient management team.

When the acknowledgment is received by patient mobile unit 102 from the network, the sent reading groups list is taken from the pending transmission list and the acknowledgment timer is removed.

As discussed above, once data has been downloaded from the glucose meter 104, server 501 commands that that data be deleted from glucose meter 104 to maintain confidentiality. Again, the data downloaded from glucose meter 104 is also stored, in a secured encrypted fashion on patient mobile unit system 102.

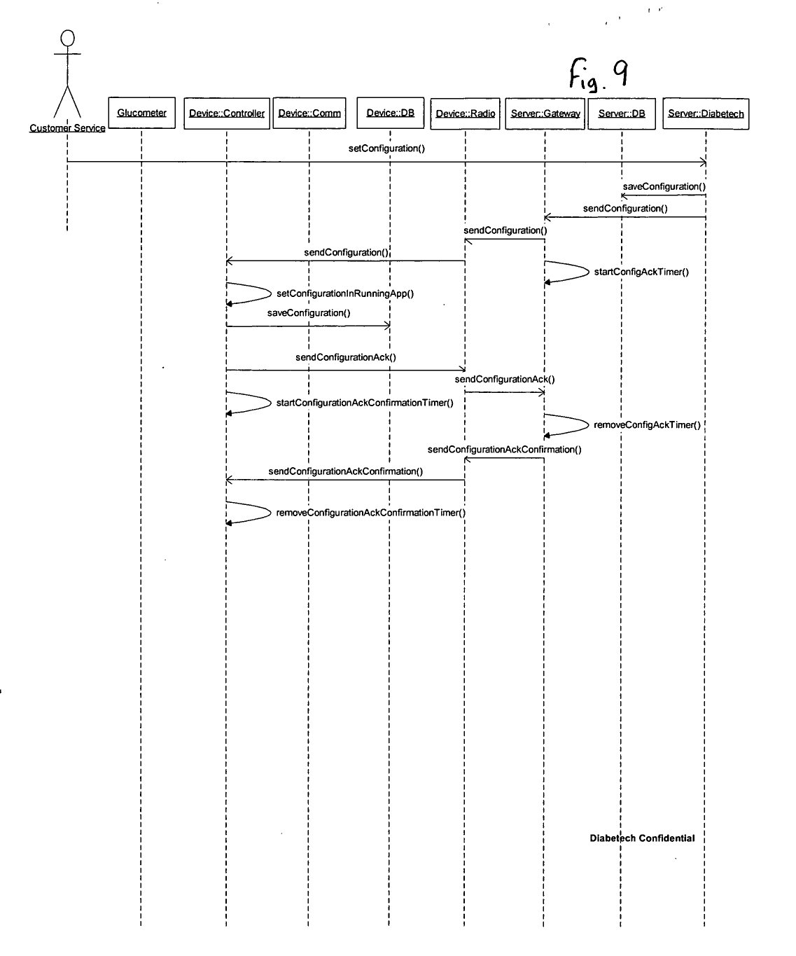

FIG. 9 is a representative sequence diagram illustrating the configuration of a given patient mobile unit 100 by server 501. In this example, a customer server's representative communicating with server 501 initiates the management unit configuration process, including entering the required configuration data. These configuration data are saved in the server database and then transmitted to the target patient mobile unit 102 via the carrier gateway 703, base station 702, and management unit 100 radio transceiver 116. Server concurrently starts a configuration acknowledgment timer setting a window during which an acknowledgment from the patient mobile unit 102 is expected.

Upon receipt of the configuration data, microprocessor 114 of patient mobile unit 102 stores those configuration data in the associated database and initiates the configuration application software. A configuration acknowledgment is then returned to server 501 and a configuration acknowledgment confirmation timer starts. Upon receipt of the configuration acknowledgment by server 501, server 501 removes the configuration acknowledgment timer and sends a configuration acknowledgment confirmation back to patient mobile unit 102. Upon receipt of the configuration acknowledgment confirmation, patient mobile unit 102 removes the configuration confirmation timer and the configuration process is complete.

Server 501 supports a number of interfaces designed to collect data from the associated patent mobile units 102, as well as subjective data from individuals regarding their health and physiologic status provided through the marking process discussed above. These data are then used by each patient and his or her diabetes management team to understand trends and the effectiveness of the standing course of treatment for that patient. Members of the medical team may also use the population management analytics to provide proactive management for large groups of patients and thereby introduce efficiencies to their practice.

In the illustrated embodiment, web-based analytics are supported through computer terminal 506. The alert feature through IVR server 504 also provides an automated window into the patient's disease state anytime and from anywhere.

As the data are collected, they can then be presented to the user via push of a report to their email, pushed stat summaries to their phone via IVR server 504, or via text messaging via text messaging pager 505. Server 501 processes the collected data using demographic data including age, gender, diabetes type, insulin therapy regimen, default meal times, various diabetes-related goals and other default behavioral factors.

One presentation of data according to the principles of the present invention. is the Triage Plot, discussed above. The Triage Plot function normalizes patient glucose levels across a potentially large group of patients and presents them on a single plot within a common view. Understanding glucose levels and averages within the context of the Normal range is much more useful than working with the raw test result. This is due to variability in the accuracy of the biometric device, for example glucose meter 104, and also the rapid change in glucose data—trends and rate of change approaching dangerous low and high levels are far more important than actual number at any given point in time.

As the classification of High, Normal and Low is a very personal assessment, it is typically not possible to classify a group within a common view and also share the H, N, L classification system across all patients. Therefore, personalized ranges of the High, Normal, and Low are identified and a specific data point determined as a percentage within one of these ranges. There is some flexibility in this plotting of ranges where one must assess relativity against no common standard.

Other parameters needed for plotting include selection of an arbitrary maximum and minimum values, such as in the case of the maximum High and the minimum Low. The upper bound of maximum should not be infinite and the lower bound of low should not be 0. Therefore, a practice may designate a minimum low across all patients as 20 in this example and a maximum high of 600. The effect on the analysis from use of the Triage Plot is effectively the same whether actual data may be 15 or in the case of a high, 1000. (Both of these data points are beyond the range of normal to the point where they would be simply classified as critical by any standard.) Another option is to personalize the extreme bounds.

Working with averages in diabetes is a fairly common in the art, and typically involves calculating average blood glucose as a simple mean across any number of data points. However, this technique can result in extremely misleading numbers as more re-tests are taken by the patient when extremely high and very low-test results are obtained. In other words, these additional tests skew the average to the extreme.

According to the principles of the present invention, tests taken within 30 minutes of each other are identified and then averaged first to account for re-testing. This pseudo data point is then used to calculate the overall average. In addition to the new pseudo data point, a pseudo timestamp is provided as an average between all of the averaged tests.

The Triage Plot, as implemented through computer terminal 506 of FIG. 5, supports a number of ease of use features for the management team member, including:
1. Trend Indicators which not only plot the point, but indicate the direction of the data trend in time.
2. A Mouse Over function allows easy focusing on individual data points in detail.
3. Multiple tool and parameter selection functions are supported within a single session.
4. A Tools Dashboard provides access to all tools in a common look and feel.
5. Data import and export support standards based schemas such as Diabet-ML, HL7 and others.
6. A Rainbow Color background provides visualization of Low, Normal and High data.
7. Multi-patient detail provides data on a single view for peer group comparison.

8. High Risk Patient Marker allows healthcare providers to flag patients at particular risk.
9. Multi-Patient Dashboard supports data evaluation for a large number of patients.

10. Individual Patient Dashboard supports focused data evaluation on a patient by patient basis.

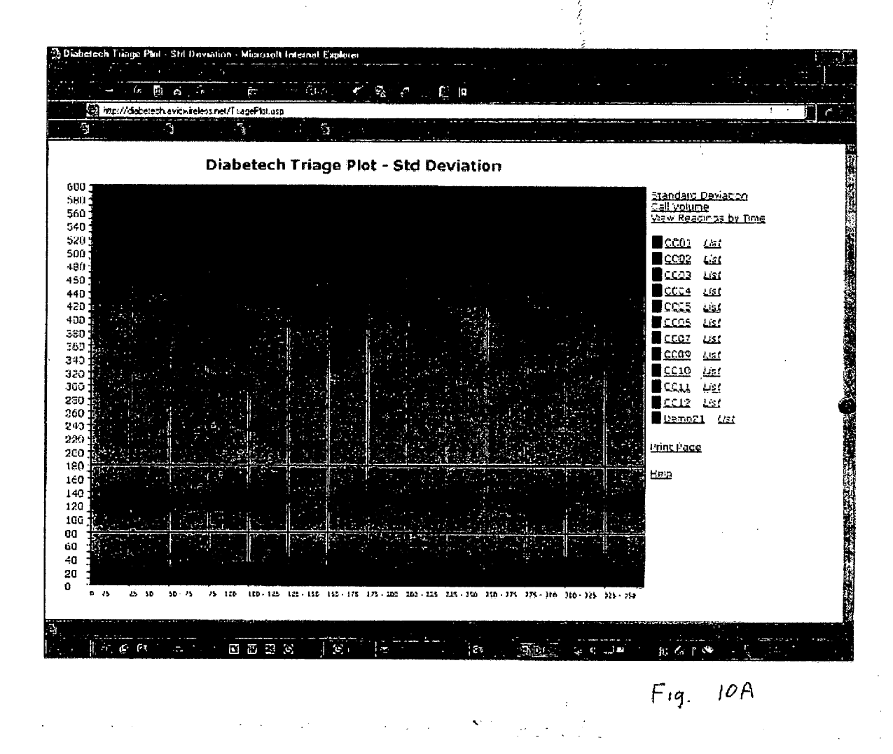

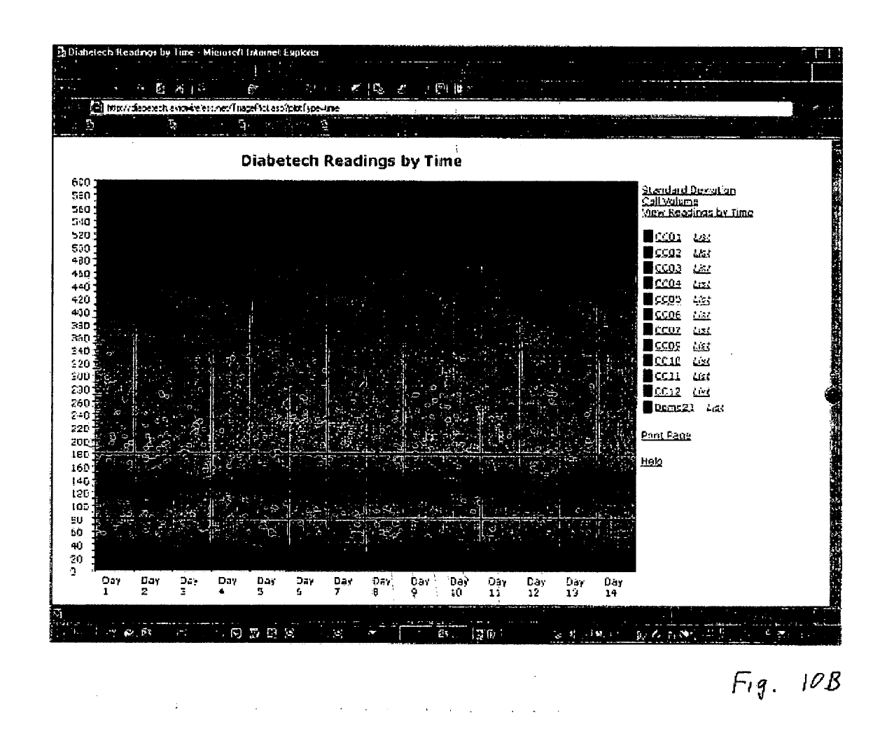

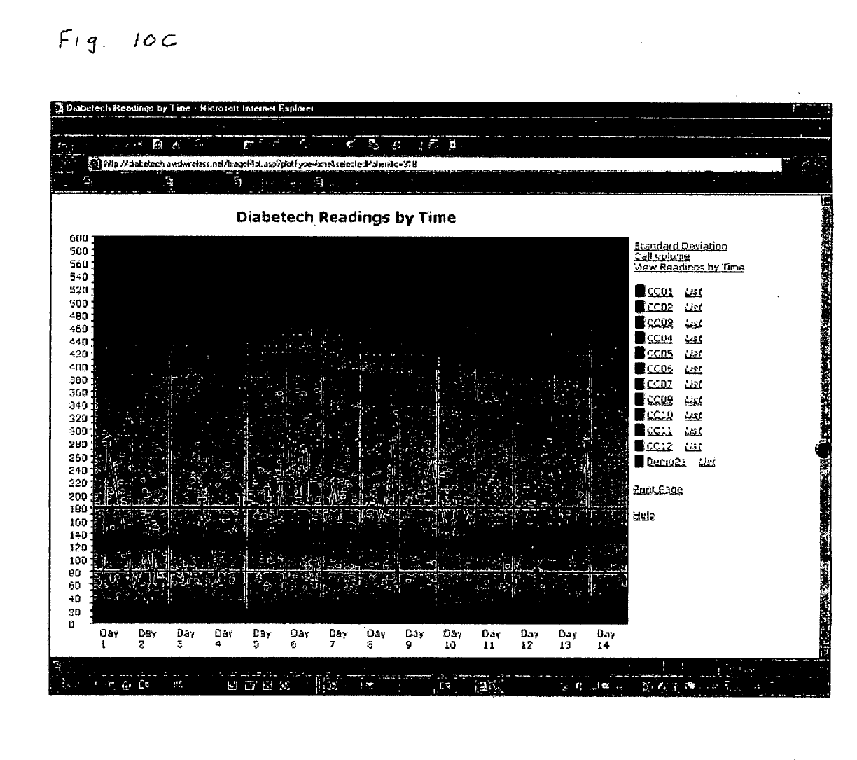

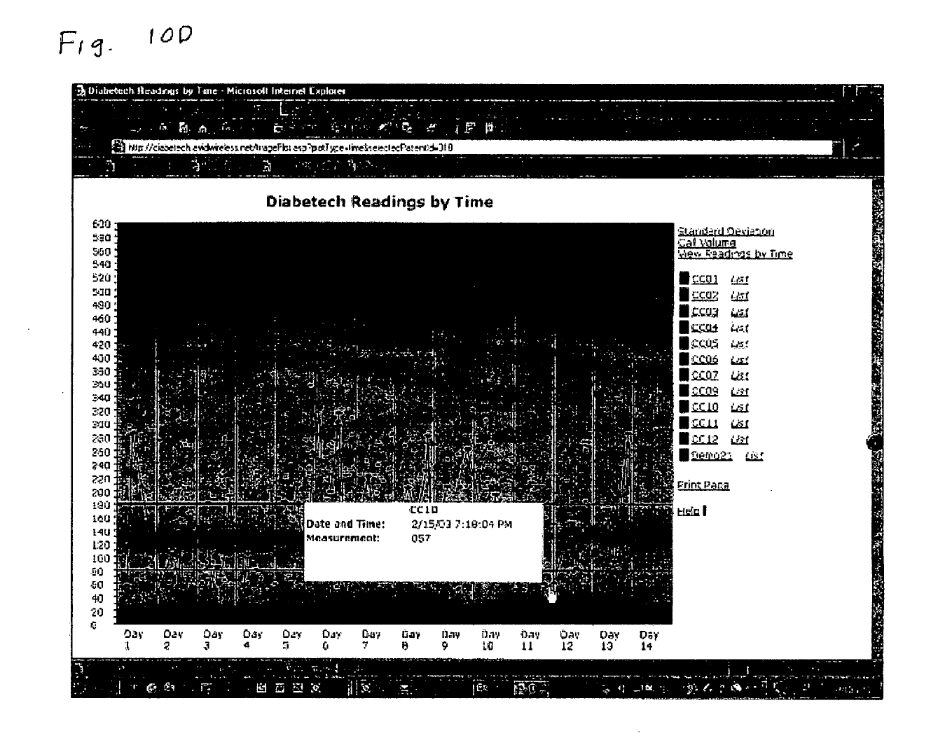

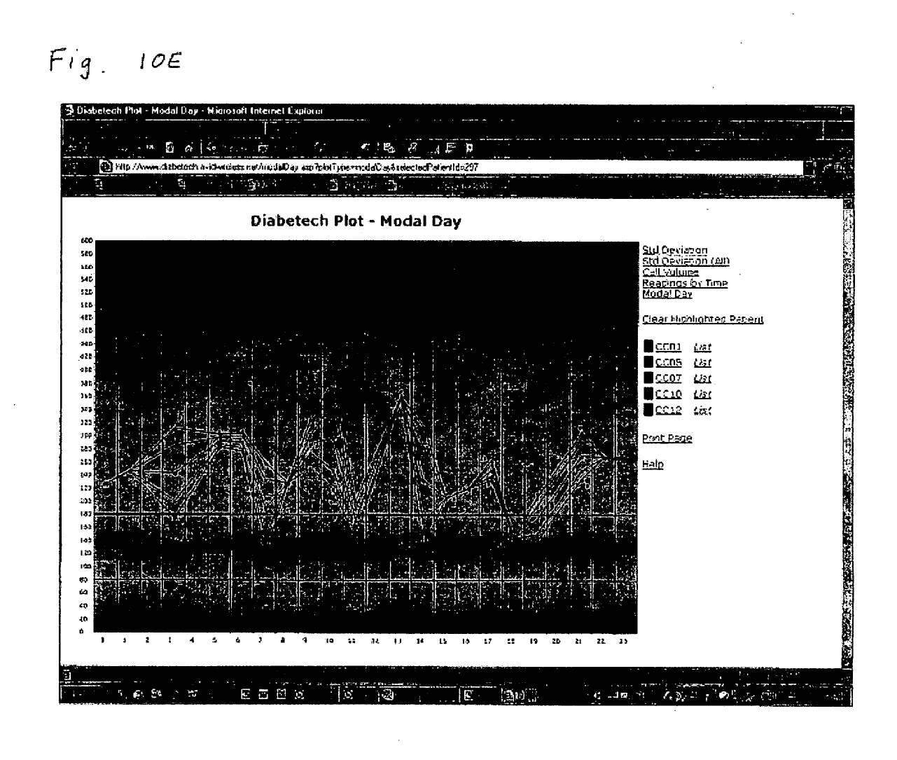

Selected features of an representative Triage Plot are provided as FIGS. 10a-10e. For purposes of illustration, the depicted Triage Plot is shown as a computer screen view, such as may be provided through computer terminal 506 and the myglucomon.com website. Alternatively, the Triage Plot can be delivered to the appropriate management team member by email, FAX, or hardcopy.

FIG. 10a illustrates a exemplary Triage Plot in which Standard Deviation and Average Blood Sugar are plotted over the 14 most recent days on a rolling basis. This Triage Plot provides at-a-Glance segmentation of potentially large patient populations. This can be an effective tool for automatically stratifying a patient population allowing the physician team to take priority action for those at most risk and poorest control.

FIG. 10b illustrates a Community Plot, which is a graphical non-patient identifiable representation of the universal database over 14 most recent days on a rolling basis. The Community Plot also provides an effective tool for automatically stratifying a patient population allowing the physician team to take priority action for those at most risk and poorest control.

A representative Patient Highlight—Rolling View of the patient self-test record over 14 most recent days on a rolling basis is shown in FIG. 10c. Specifically, FIG. 10C shows a graphical representation of the log data log for the selected patient. The Mouse-over of Patient Highlight feature, which allows easy identification from the logged data of any data point on the plot, is illustrated in FIG. 10d.

FIG. 10e is an exemplary Modal Day Plot, which shows readings for a single patient from the last 14 days in a modal day view. In particular, the readings are plotted by hour of day (0-23:00 hours). Trend lines for 10th, 25th, 50th, 75th and 90th percentiles are plotted as well. Clicking on a patient name on the right will change the Modal Day to that patient.

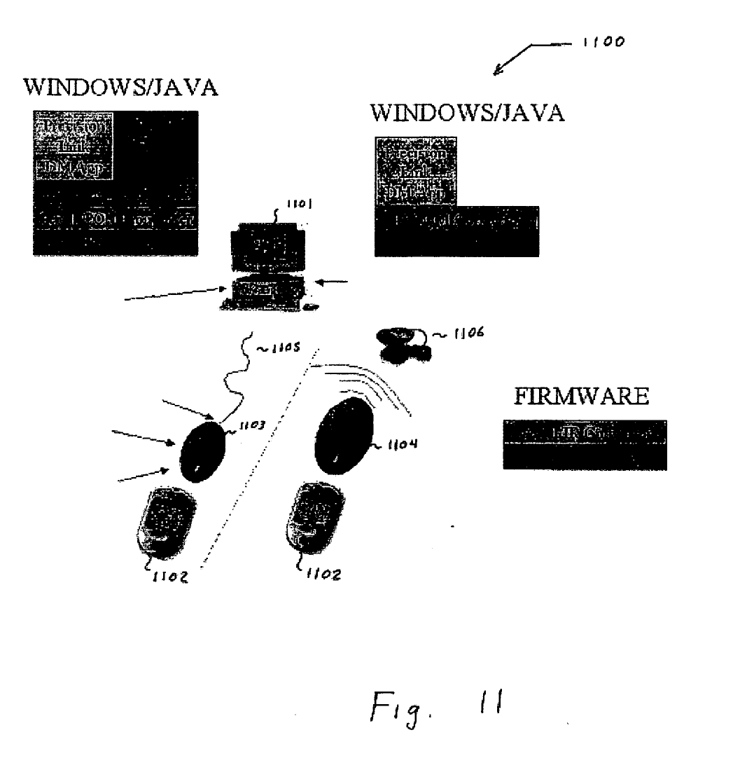

A home monitoring and goaling system 1100 according to another embodiment of the principles of the present invention is shown in FIG. 11. Advantageously, a patient or patient management team member can set a goal, such as a target number of biometric tests to be taken by the patient, and then compliance with that goal monitored using a computer terminal 1101, or other information storage and display device. For example, for a child patient, positive feedback in the form of audible or visual presentations may be used as a reward and encouragement for meeting the target. For both adults and children, the recorded information may be exchanged with other peers, using email or a common website for example, such that peers within a given group can provide encouragement and support among themselves.

System 1100 can be implemented in a number of ways. In each case, a biometric device 1102, such as a glucose meter in the case of diabetes, is used by the patient to perform the actual test. The resulting test data is then passed to an interface pod 1103 or 1104, depending on whether a wireless or hardwired connection to computer terminal 1101 is being utilized. In the case of a wired connection, pod 1103 preferably couples with computer terminal 1101 through a universal serial bus (USB) 1005. Advantageously, USB 1105 allows pod 1003 to be powered directly from computer terminal 1101. In the case of a wireless connection, an infrared (1R) port associated with computer terminal 1101 provides the communications link with pod 1104. Here, pod 1104 requires a self-contained power source, such as a battery.

Each pod 1103 and 1104 includes firmware providing the communications (COM) interface with the corresponding biometric device 1102, as well as the selected hardwired—serial or wireless interface with computer terminal 1101. Computer terminal 1101 maintains the software required to interface with the desired wired (USB) or wireless (IR) link and software such as the commercially available Precision Data Link Data Management software for interpreting and presenting the data extracted from biometric device 1102. Goaling software allows the patient, a management team member, or peer group member to set target goals and monitor progress towards those goals.

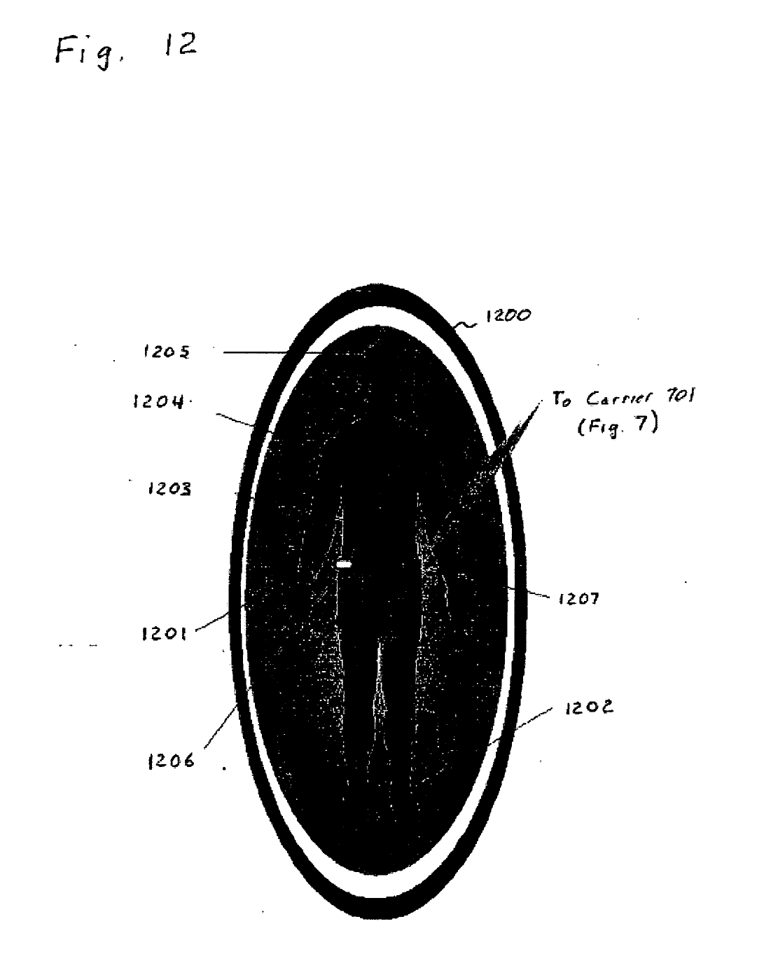

The principles of the present invention, demonstrated above with respects to patient management unit 100 and the system of FIGS. 5 and 6, are extended as shown in FIG. 12 to a system 1200 in which the biometric sensors and patient mobile unit are either worn on-body or kept close to the body. Advantageously, a patient, or other wearer being monitored, such as an athlete or soldier, can be automatically and continuously monitored with minimal, if any, user intervention.

In the embodiment shown in FIG. 12, system 1200 includes a local management device 1201, in this case in the form of a wrist watch, which receives input data from a set of sensors monitoring various body functions. In this example, a set of commercially available sensors includes an accelerometer 1202, a continuous glucose sensor 1203, and ECG/blood pressure sensor 1204, and a thermometer 1205 are shown for reference. The number and type of sensors will vary however depending on the application for which system 1200 is intended. Communications between the management unit 1201 and the sensors 1202-1205 is preferably established using short range radio, although hardwired embodiments are also possible.

In response to the set of sensors, management unit 1201 controls an on- or near-body medical delivery device, in this example an insulin pump 1206. In the illustrated embodiment, insulin pump 1206 communicates with management unit 1201 via a short range radio link. Thus, in the present example, system 1200 allows for automatic control of the wearers insulin level with minimal intervention. In alternate embodiments, the medical deliver device may vary, depending on the type of medical condition being addressed and the medication required.

A telemetry module 1207 allows management unit 1201 to transmit data concerning the wearer of system 1200 to a central processing node, such as server 501 of the system shown in FIG. 5. As discussed above, the data received by the central processing node can then be used by a management team to monitor the wearer's medical condition, watch for trends, or take appropriate action in the event a critical or emergency condition has arisen. Also as discussed above with respects to server 501, the central processing node can be used by the management team to send commands and configuration data to management unit 1201 in order to precisely control the monitoring and management regimen of the wearer.

In the preferred embodiment, telemetry module includes a radio unit which operates in short, medium, and long range modes. The short range mode is primarily utilized to support communications between telemetry module 1207, management unit 1201, and/or sensors 1202-1205. The medium range mode is primarily utilized for establishing a connection to a wireless access point, such as an IEEE 802.11x access point, and in turn communications with a local or global computer network. The long range mode supports communications with a wireless carrier, such as carrier 701 discussed above. Hence, system 1200 allows a choice in the communications link between the monitored wearer and the management team based on such factors as availability, reliability, bandwidth, and cost, among other things.

The embodiments and examples set forth herein are presented to best explain the present invention and its practical application and to thereby enable those skilled in the art to make and utilize the invention. Those skilled in the art, however, will recognize that the forgoing description and examples have been presented for the purpose of illustration and example only. Other variations and modifications of the present invention will be apparent to those of skill in the art. The description as set forth is not intended to be exhaustive to limit the scope of the invention. It is contemplated that the use of the present invention can involve components having different characteristics.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore, contemplated that the claims will cover any such modifications or embodiments that fall within the true scope of the invention.

1. An system for remotely monitoring a medical condition of a patient comprising:
an input device for inputting test information for evaluating a selected medical condition of a patient;
a communications device for selectively transmitting test information received from the input device; and
a base system for receiving the test information from the communications device for distribution to at least one member of a medical condition management team.

2. The system of claim 1, wherein the selected medical condition is diabetes.

3. The system of claim 1, wherein the communications device transmits information at least in part via a wireless telecommunications system.

4. The system of claim 1, wherein the communications device transmits information at least in part via a global computer network.

5. The system of claim 1, wherein the base system distributes information to the at least one member of the medical condition management team via a telecommunications system.

6. The system of claim 1, wherein the base system distributes information to the at least one member of the medical condition management team via a global computer network.

7. The system of claim 6, wherein the base system distributes information via a global computer terminal in the form of a triage plot, the triage plot statistically presenting information received from a corresponding number of input devices.

8. The system of claim 1, wherein the input device forms a portion of a patient transportable unit.

9. The system of claim 1, wherein the input device comprises a desktop unit operating in conjunction with a computer terminal.

10. A chronic disease management system comprising:
a server for processing information being exchanged between a patient and a patient management team;
a patient interface for exchanging information with the server including selected medical condition information input by the patient; and
a management team interface for exchanging information between the patient management team and the server including information input by a member of the patient management team for controlling the patient interface and for processing information received from the patient interface by the server.

11. The system of claim 10, wherein the management team interface comprises an interactive voice response system.

12. The system of claim 10, wherein the management team interface comprises a website.

13. The system of claim 10, wherein the management team interface supports marking of information received by the server from the patient interface.

14. The system of claim 10, wherein the management team interface allows a member of the patient management team to selectively activate and deactivate the patient interface.

15. The system of claim 10, wherein the management team interface allows a member of the patient management team to statistically analyze information received from a plurality of patient interfaces communicating with the server.

16. The system of claim 10, wherein the server is operable to automatically send alerts to a patient management team member in response to medical condition information of selected parameters received from the patient interface.

17. The system of claim 10, wherein the patient interface is supported by a transportable patient mobile unit communicating with the server at least in part by a wireless communications link.

18. The system of claim 10, wherein the patient interface is supported by a desktop hardware interface coupled to a computer terminal.

19. The system of claim 18, wherein the computer terminal supports goaling.

20. The system of claim 19, wherein the computer terminal supports peer goaling.
